(19) United States
(12) Patent Application Publication
(10) Pub. No.: US 2003/0069187 A1
(43) Pub. Date: Apr. 10, 2003

(54) Elastase inhibitors

(76) Inventors: Srinivasa K. Rao, New Hyde Park, NY (US); Mahesh Mathrubutham, Forest Park, IL (US); Leslie Kushner, Forest Hills, NY (US); Glenn R. Faust, Roslyn, NY (US)

Correspondence Address: Elie H. Gendloff, Ph.D., Esq., AMSTER, ROTHSTEIN & EBENSTEIN, 90 Park Avenue, New York, NY, 10016, US

(21) Appl. No.: 09/972,843

(22) Filed: Oct. 5, 2001

(51) Int. Cl. A61K 38/10; A61K 38/08; C12Q 1/68; G01N 33/53; G01N 33/567; C07K 7/08; C07K 7/06

(52) U.S. Cl. 514/16; 435/6; 435/7.2; 530/329

(57) ABSTRACT

Naturally occurring novel elastin derived oligopeptides, having the formula AFP, VPG and AGIP, are disclosed. The oligopeptides are strong competitive inhibitors of elastolytic serine proteases, particularly neutrophil elastases. The novel oligopeptides are useful in methods of inhibiting elastolytic serine protease and methods of detecting a disease characterized by elevated activity of elastolytic serine proteases. Methods for determining the severity of diseases characterized by elevated activity of elastolytic serine proteases are also disclosed.

### BACKGROUND OF THE INVENTION

(1) Field of the Invention

The present invention generally relates to enzyme inhibitors. More particularly, the invention is directed to inhibitors of elastolytic serine proteases, and diagnostic and therapeutic applications using the inhibitors. The inhibitors are small elastin derived peptides.

(2) Description of Related Art

References cited

Akers et al., 1992, Am. Rev. Respir. Dis. 145:1077-1081.

Bonnaure-Mallet et al., 1995, J. Periodontal Res. 30:58-65.

Broch et al., 1998, J. Biomol. Struct. Dyn. 15:1073-1091.

Brassart et al., 2001, J. Biol. Chem. 276:5222-5227.

Castiglione Morelli et al., 1997, J. Pept. Res. 49:492-499.

Cohen et al., 1992, Anal. Surg. 216:327-332.

Cohen et al., 1993, Cardiovasc. Surg. 1:373-376.

Cowan et al., 2000, Nat. Med. 6:698-702.

Crombie et al., 1973, Biochem. Biophys. Res. Commun. 52:1228-1233.

Dillon et al., 1992, Am. Rev. Respir. Dis. 146:1143-1148.

Edwards et al., 1994, Med. Res. Rev. 14:127-194.

Hamato et al., 1995, J. Biochem. (Tokyo) 117:432-437.

Juvonen et al., 1994, Ann. Chir. Gynaecol. 83:296-302.

Kamoun et al., 1995, Cell Adhes. Commun. 3:273-281.

Kieber-Emmons et al., 1997, Curr. Opin. Biotechnol. 8:435-441.

Kucich et al., 1983, Am. Rev. Respir. Dis. 127:S28-S30.

Kucich et al., 1991, Am. Rev. Respir. Dis. 143:279-283.

Lineweaver et al., 1934, J. Am. Chem. Soc. 56:658-666.

Mathrubutham et al., 1998, Clin. Chem. 44:664-666.

Mehdi et al., 1990, Biochem. Biophys. Res. Commun. 166:595-600.

Nogami et al., 2000, Eur. J. Pharmacol. 390:197-202.

Orita et al., 1995, J. Antibiot. (Tokyo) 48:1430-1434.

Petersen et al., 2001, Eur. J. Vasc. Endovasc. Surg. 22:48-52.

Rao et al., 1996, Ann.NY.Acad.Sci. 800:131-137.

Rao et al., 1997, Anal. Biochem. 250:222-227.

Ripka et al., 1998, Curr. Opin. Chem. Biol. 2:441-452.

Sanderson, 1999, Med. Res. Rev. 19:179-197.

Sarfati et al., 1996, J. Surg. Res. 61:84-98.

Senior et al., 1984, J. Cell. Biol. 99:870-874.

Shinguh et al., 1998, Eur. J. Pharmacol. 345:299-308.

Stone et al., 1990, Am. Rev. Respir. Dis. 141:47-52.

de la Sierra et al., 1990, J. Mol. Recognit. 3:36-44.

Tyagi et al., 1993, J. Biol. Chem. 268:16513-16518.

Tyagi et al., 1994, Biochem. Cell. Biol. 72:419-427.

White et al.,1993, J. Vasc. Surg. 17:371-381.

Yamamoto et al., 2000, Eur. J. Pharmacol. 409:179-183.

Yasumuro et al., 1995, J. Antibiot. (Tokyo) 48:1425-1429.

Yavin et al., 1998, J. Pept. Res. 51:282-289.

U.S. Pat. No. 6,001,814.

U.S. Pat. No. 6,001,813.

U.S. Pat. No. 6,150,334.

U.S. Pat. No. 6,001,811.

Neutrophil elastase and other elastolytic serine proteases are implicated as factors in a number of diseases where activity of these enzymes is elevated. Examples of diseases where elastolytic serine proteases are elevated are abdominal aortic aneurysm, adult respiratory distress syndrome, septic shock, chronic obstructive pulmonary disease, pulmonary emphysema, multiple organ failure and pulmonary hypertension (Rao et al, 1996; U.S. Pat. No. 6,001,814). Human neutrophil elastase is also implicated in the outcome of other diseases and conditions, including arthritis, periodontal disease, glomerulonephritis, dermatitis, psoriasis, cystic fibrosis, chronic bronchitis, atherosclerosis, Alzheimer's disease, organ transplantation, corneal ulcers, and invasion behavior of malignant tumors (see, e.g., U.S. Pat. No. 6,001,814). Elastolytic digestion of elastin generates elastin derived peptides (EDP), which are often elevated in subjects having the above diseases (Akers et al., 1992; Kucich et al., 1983; Cohen et al., 1993; Kucich et al, 1991; Dillon et al., 1992). The EDPs themselves have a number of biological activities that can affect disease progression. These activities variously include chemotaxis of monocytes and fibroblasts, stimulation of fibroblast growth, and inhibition of elastolytic serine proteases (Senior et al., 1984; Broch et al., 1998; Crombie et al., 1973; Cohen et al., 1992; Brassart et al., 1995; Bonnaure-Mallet et al., 1995; Komoun et al., 1995; Juvonen et al., 1994; Castiglione Morelli et al., 1997; Sarfati et al., 1996; Tyagi et al., 1994). The inhibitor activity implicates some EDPs in negative feedback regulation of the proteases (Tyagi et al., 1993).

An example of a disease where neutrophil elastase plays an important role is abdominal aortic aneurysm (AAA) (Rao et al., 1996). Certain chemotactic EDPs increase protease activity at the site of inflammation by attracting inflammatory cells to the site of inflammation (Senior et al., 1984; Cohen et al., 1992; Brassart et al., 1995) and accelerating AAA growth. AAA growth is generally a slow process, but the presence of chemotactic EDPs accelerates its progression. Thus, treatment with elastase inhibitors would be expected to prevent generation of chemotactic EDPs and slow progression of the disease.

Several inhibitors of elastolytic serine proteases in general and neutrophil elastase in particular have been discovered (Shinguh et al., 1998; Yasumuro et al., 1995; Hamato et al., 1995; Yamamoto et al., 2000; Cowan et al., 2000; Nogami et al., 2000; Orita et al., 1995; Yavin et al., 1998; de la Sierra et al., 1990; Mehdi et al., 1990; Stone et al., 1990; Edwards et al., 1994; U.S. Pat. No. 6,001,814; U.S. Pat. No. 6,001,813; U.S. Pat. No. 6,150,334; U.S. Pat. No. 6,001,811). These inhibitors are generally not naturally occurring or have relatively low activity. Thus, there is a need for inhibitors of elastolytic serine proteases, particularly neutrophil elastases, that are naturally occurring and have high inhibitory activity. Such inhibitors would be useful for therapeutic and diagnostic purposes. Naturally occurring inhibitors would also be useful to elucidate the innate mechanisms involved in regulation of elastolytic serine protease activity. The present invention provides three novel inhibitor oligopeptides to satisfy that need.

### BRIEF SUMMARY OF THE INVENTION

The present invention is directed to the discovery and use of three novel oligopeptides, Ala-Phe-Pro (AFP), Val-Pro-Gly (VPG), and Ala-Gly-Ile-Pro (AGIP), which are inhibitors of elastolytic serine proteases, particularly neutrophil elastase. Methods for using the peptides diagnostically and therapeutically are also disclosed.

Thus, in one aspect, the present invention is directed to an isolated and purified oligopeptide. The oligopeptide consists of 8 or less amino acids or amino acid mimetics and comprises the amino acid or mimetic sequence AFP.

In additional embodiments, the invention is directed to an isolated and purified oligopeptide, consisting of the amino acid or mimetic sequence VPG.

In another aspect, the invention is directed to an isolated and purified oligopeptide, consisting of about 20 or less amino acids or amino acid mimetics and comprising the amino acid or mimetic sequence AGIP.

Additionally, the present invention is directed to a method of inhibiting an elastolytic serine protease. The method comprises contacting the elastolytic serine protease with an oligopeptide, wherein the oligopeptide consists of about 50 or fewer amino acids or amino acid mimetics, and wherein the oligopeptide comprises an amino acid or mimetic sequence selected from the group consisting of AFP, VPG and AGIP.

The present invention is also directed to a method of detecting a disease in a mammal, wherein the disease is characterized by elevated activity of an elastolytic serine protease. The method comprises determining whether the mammal has one of the peptides AFP, VPG or AGIP.

In additional embodiments, the present invention is directed to a method for determining the severity of a disease in a mammal, wherein the disease is characterized by elevated activity of an elastolytic serine protease. The method comprises determining the concentration of one of the peptides AFP, VPG or AGIP. In this method, a concentration of the peptide below the concentration found in a mammal with a mild case of the disease indicates that the mammal has a severe case of the disease.

### BRIEF DESCRIPTION OF THE DRAWING

FIG. 1 is a bar graph depicting results from experiments in which the inhibitory capacity of each EDP isolate was evaluated in triplicate using 200 μg of succinylated elastin (Rao et al., 1997; Mathrubutham et al., 1998), 30 μg of Total EDP, 3 mg of G-25 fractions and RP-HPLC fractions and 0.029 U PPE in 150 μl with 50 mM sodium borate pH8.5. Reactions were incubated at 37° C. for 30 minutes, then treated with 2,4,6-trinitrobenzene sufonic acid. Absorbance at A₄₅₀ₙₘ was determined after 20 min at room temperature. Elastase inhibitory capacity was evaluated by comparing the activity of PPE in the presence and absence of EDP, where elastase in the absence of EDP was considered to represent 100% elastase activity. The data was standardized to protein content and is presented as % inhibition per μg of protein. VGVAPG is an EDP that is chemotactic for inflammatory cells (Senior et al., 1984) and was assayed for elastase inhibition to demonstrate that elastase inhibition is a characteristic that is not shared by all EDPs. PEP7 is VPG; PEP8 is AGIP and PEP9 is AFP.

### DETAILED DESCRIPTION OF THE INVENTION

The present invention is based on the discovery that three oligopeptides, Ala-Phe-Pro (AFP), Val-Pro-Gly (VPG), and Ala-Gly-Ile-Pro (AGIP), are naturally occurring elastin derived peptides (EDP) which are strong competitive inhibitors of elastolytic serine proteases. These oligopeptides are derived by elastase digestion of elastin. They are fragments of a previously known EDP, of MW 57kD (Bonnaure-Mallet et al., 1995), that also has elastase inhibitory activity. The inhibitory activity of the oligopeptides of the present invention is about 60-fold that of the 57 kD EDP, however (Example 1).

As is understood by the skilled artisan, serine proteases are a large group of endoproteases that have a reaction mechanism based on the formation of an acyl-enzyme intermediate on a specific active serine residue. Some serine proteases, the elastolytic serine proteases, have activity on elastin. It is believed that the three oligopeptides of the present invention have inhibitory activity on all elastolytic serine proteases.

Thus, in some embodiments, the present invention is directed to an isolated and purified oligopeptide including peptidomimetics comprising the amino acid or mimetic sequence AFP. Since this peptide has elastase inhibitory activity and is a fragment of the 57 kD peptide, which also has elastase inhibitory activity, it is likely that these oligopeptide sequences impart inhibitory activity to the 57 kD EDP. This indicates that the oligopeptides of this invention would be effective inhibitors as a portion of a larger oligopeptide or polypeptide. Therefore, the skilled artisan would expect that any oligopeptide containing the sequence AFP would have elastase inhibitory activity. Preferably, the AFP-containing oligopeptide of this invention consists of 8 or less amino acids or mimetics. In more preferred embodiments, the oligopeptide consists of less than 8 amino acids or mimetics, and can consist of 7, 6, 5 or 4 amino acids or mimetics. In the most preferred embodiments, the oligopeptide consists of only the amino acid or mimetic sequence AFP.

As used herein, an amino acid mimetic or peptidomimetic is a compound containing non-peptidic structural elements, wherein the compound is capable of mimicking or antagonizing the biological action(s) (here, inhibitory activity) of a natural parent peptide. Peptidomimetics are generally not substrates of proteases and are likely to be active in vivo for a longer period of time as compared to the peptides. In addition, they might be less antigenic and might show an overall higher bioavailability. The skilled artisan would understand that design and synthesis of peptidomimetics that could substitute for any particular oligopeptide (such as the inhibitors of this invention) would not require undue experimentation. See, e.g., Ripka et al., 1998; Kieber-Emmons et al., 1997; Sanderson, 1999.

As used herein, an oligopeptide is a peptide of about 50 or fewer amino acids. Included herein are peptides with unusual amino acids incorporated, as well as peptidomimetics. The amino acids or amino acid mimetics can also have other compounds attached, such as saccharides, dyes, haptens, etc., provided those compounds do not interfere with the inhibitory activity of the oligopeptide. Several well known methods are available to synthesize any oligopeptide without undue experimentation.

In other embodiments, the invention is directed to an isolated and purified oligopeptide, consisting of the amino acid or mimetic sequence VPG.

Additionally, the present invention provides an isolated and purified oligopeptide, consisting of about 20 or less amino acids or amino acid mimetics and comprising the amino acid or mimetic sequence AGIP. In more preferred embodiments, the oligopeptide consists of fewer than 20 amino acids or mimetics, and can consist of 19, 18, 17, 16, 15, 14, 13, 12, 11, 10, 9, 8, 7, 6 or 5 amino acids or mimetics. In the most preferred embodiments, the oligopeptide consists of only the amino acid or mimetic sequence AGIP.

The oligopeptides described above are competitive inhibitors to elastolytic serine proteases. Thus, the invention provides a method of inhibiting an elastolytic serine protease. The method comprises contacting the elastolytic serine protease with an oligopeptide, wherein the oligopeptide consists of about 50 or fewer amino acids or amino acid mimetics, and wherein the oligopeptide comprises an amino acid or mimetic sequence selected from the group consisting of AFP, VPG and AGIP. In preferred embodiments the oligopeptides are of shorter length, for example 45, 40, 35, 30, 25, 20, 15, 10, or 5 amino acids or mimetics in length. In more preferred embodiments, the oligopeptide consists of AFP, VPG or AGIP. In the most preferred embodiments, the oligopeptide consists of AFP, since that peptide is the most inhibitory.

While the oligopeptides of the present invention are expected to be inhibitory to any elastolytic serine proteases, they are believed to be the most inhibitory to neutrophil elastase. Thus, in preferred embodiments, the method is used to inhibit a neutrophil elastase. Also, since neutrophil elastase is implicated in various human diseases, it is preferred that the neutrophil elastase of this method is a human neutrophil elastase, although the oligopeptides of the invention would be expected to inhibit any mammalian neutrophil elastase.

The method for inhibiting a elastolytic serine protease described above is useful for inhibiting those proteases in vitro, as well as in a living mammal. Preferably the living mammal is a human and the elastolytic serine protease is neutrophil elastase. In these embodiments, the mammal/human may be suffering from, or at risk for, a disease characterized by elevated activity of an elastolytic serine protease, preferably neutrophil elastase. Non-limiting examples of such diseases are abdominal aortic aneurysm (AAA), adult respiratory distress syndrome, septic shock, chronic obstructive pulmonary disease, pulmonary emphysema, and pulmonary hypertension. In preferred embodiments, the disease is AAA.

It is also envisioned that the inhibitory peptides of the invention could be used to diagnose diseases that are characterized by elevated activity of an elastolytic serine protease. Elevated EDPs have been previously found to be associated with chronic obstructive pulmonary disease (Akers et al., 1992; Kucich et al., 1983), abdominal aortic aneurysms (Cohen et al., 1993) and emphysema (Kucich et al, 1991; Dillon et al., 1992). Additionally, differences in severity of abdominal aortic aneurysms have been associated with differences in EDP levels, with more severe disease correlating with lower EDP concentrations (Petersen et al., 2001). The latter finding suggests that higher levels of EDPs that inhibit elastase would prevent the inhibited elastase from exacerbating the disease. Thus, applying those findings to the instant invention, the presence of the inhibitory peptides AFP, VPG and/or AGIP would indicate that elastin is being digested by an elastolytic serine protease, indicating the presence of disease, whereas higher levels of the inhibitory peptides would indicate that the elastases are being inhibited from further digesting elastin, indicating that progression to more severe disease is not likely. The particular levels of the inhibitory peptides that indicate rapid or slow progression of the disease could be determined by the skilled artisan without undue experimentation.

Thus, the present invention provides a method of detecting a disease in a mammal, wherein the disease is characterized by elevated activity of an elastolytic serine protease. The method comprises determining whether the mammal has one of the oligopeptides AFP, VPG or AGIP. In preferred embodiments of this method, the mammal is a human, and elastolytic serine protease is a neutrophil elastase. Preferred diseases useful for this method are AAA, adult respiratory distress syndrome, septic shock, chronic obstructive pulmonary disease, pulmonary emphysema, and pulmonary hypertension; the most preferred disease is AAA.

The determination of whether the mammal has one of the inhibitory oligopeptides can be made by any means appropriate for the disease. For example, with pulmonary diseases such as chronic obstructive pulmonary disease, pulmonary emphysema, and pulmonary hypertension, the sputum would likely have the highest level of the inhibitory oligopeptides. Conversely, for AAA, the highest level of the inhibitory oligopeptides would likely be in the blood plasma (as in Petersen et al., 2001) or urine. Thus, in preferred embodiments, the determination is made by testing a bodily fluid of the mammal for the peptide, where examples of bodily fluids are sputum, urine, and plasma. The presence of the peptides is preferably determined by chromatographic methods, for example by G-25 Sephadex size fractionation followed by high performance liquid chromatography (HPLC). See Example.

In additional embodiments, the invention is directed to a method for determining the severity of a disease in a mammal, wherein the disease is characterized by elevated activity of an elastolytic serine protease. The method comprises determining the concentration of at least one of the oligopeptides AFP, VPG or AGIP. In these embodiments, a concentration of the peptide below the concentration found in a second mammal with a mild case of the disease indicates that the mammal has a severe case of the disease. Preferably, the mammal is a human and the elastolytic serine protease is a neutrophil elastase. Preferred diseases for this method are AAA, adult respiratory distress syndrome, septic shock, pulmonary emphysema, and pulmonary hypertension; the most preferred disease is AAA. As with the previously described method, the determination is preferably made by measuring the concentration of the oligopeptide in a bodily fluid of the mammal, such as sputum, urine or plasma. The concentration of the peptides is preferably determined by chromatographic methods, for example by G-25 Sephadex size fractionation followed by HPLC.

Preferred embodiments of the invention are described in the following examples. Other embodiments within the scope of the claims herein will be apparent to one skilled in the art from consideration of the specification or practice of the invention as disclosed herein. It is intended that the specification, together with the examples, be considered exemplary only, with the scope and spirit of the invention being indicated by the claims which follow the examples.

### EXAMPLE 1.

### Isolation and Characterization of Novel Human Neutrophil Elastase Inhibitors

These studies establish that naturally occurring inhibitory EDPs exist that are fragments of the 57 kD inhibitory EDP (Bonnaure-Mallet et al. 1995).

Materials and Methods

Total EDP was generated by digesting 100 mg of insoluble bovine neck ligament elastin (EPC, Owensville, Mo.) with 27.8 Units (U) of porcine pancreatic elastase (PPE; Calbiochem, San Diego, Calif.) in 50 mM sodium borate pH 8.5, 0.1 M NaCl, 0.05 M Tris pH 7.5, 0.025% Triton X-100, and incubated at 37° C. for 24 hrs. The digest was then centrifuged at 14000 rpm for 2 min and the supernatant was collected.

Total EDP was fractionated on a G-25 Sephadex column equilibrated with 50 mM sodium borate pH 8.5 at a flow rate of 0.7 ml/minute. G-25 inhibitory fractions were then analyzed by reverse phase high performance liquid chromatography (RP-HPLC) using a C-18 column (Vydac, Hesperia, Calif.) with 5% acetonitrile, 0.1% triflouroacetic acid pH 2.3 as RP buffer at a flow rate of 0.5 ml/minutes and monitored at A₂₀₅ₙₘ. Fractions were collected, dried, and resuspended in 50 μl water and assayed for elastase inhibitory capacity (Rao et al., 1997; Mathrabutham et al., 1998). The inhibitory capacity of G-25 and RP-HPLC EDP fractions were evaluated (Rao et al., 1997; Mathrabutham et al., 1998) using 3 μg of G-25 fraction and RP-HPLC peptide with 0.029 U PPE. Percentage inhibitory capacity was evaluated by comparing the activity of PPE in the presence and absence (100% activity) of EDP and standardized to μg protein. The inhibitor constant (Ki) was experimentally calculated (Lineweaver et al., 1936). The assay was carried out in triplicate as previously described (Rao et al., 1997) using 11.16-134 μM elastin substrate, 3.34-9.94 μM AFP peptide, 0.029 U of porcine pancreatic elastase (PPE) and 0.010 U human neutrophil elastase (HNE; 21 U/mg; Calbiochem San Diego, Calif.) and incubated at 37° C. for 1,3 and 5 min. Ki is a measure of inhibiting effect and lower Ki indicates greater inhibition.

Results and Discussion

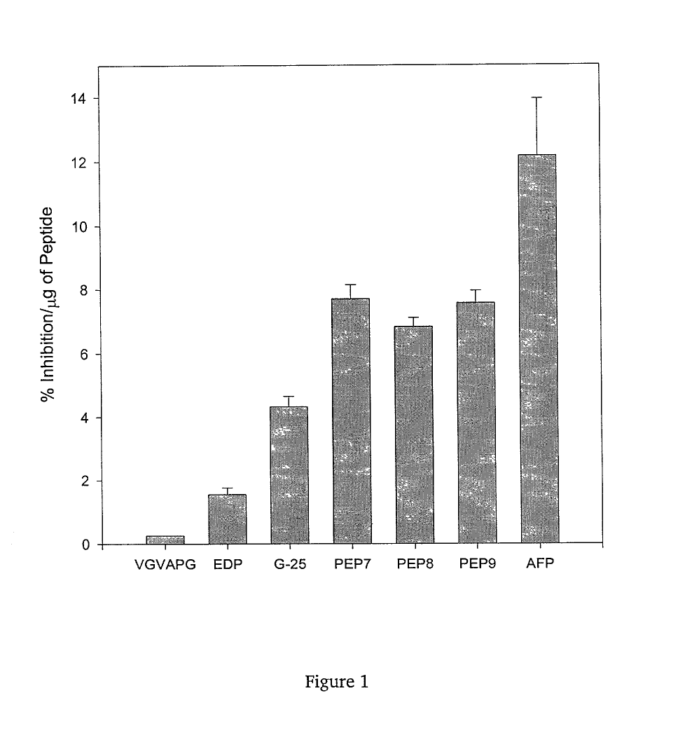

G-25 Sephadex fractionation identified EDPs that had a 2.7-fold increase in elastase inhibitory capacity over Total EDP (p<0.001). Further RP-HPLC analysis identified three peptides (VPG, AGIP, and AFP—PEP7, PEP8, and PEP9 of FIG. 1) that had 4.8, 4.3 and 4.4-fold increase, respectively, in elastase inhibitory capacity compared to total EDP (p<0.001). Molecular modeling (to visualize the peptide interaction with HNE) indicated that, of the three peptides, AFP docked with the active site of HNE, suggesting specific inhibition of HNE. Synthetic AFP had a 7.6-fold increase in elastase inhibitory capacity over total EDP (p<0.001) (FIG. 1). Kinetic analysis indicated that AFP is a competitive inhibitor of PPE (Ki=38 nM) and HNE (Ki=23 nM).

Degradation of aortic wall elastin is an initial step in AAA development (White et al., 1993), which generates EDPs that may contribute to elevation in proteolytic activity. However, AAA development is a slow process and we propose that even though some EDPs may promote elevated proteolytic activity at the site of inflammation, there is a subset of small EDPs that act to inhibit protease activity and reduce connective tissue degradation. This results in a dynamic balance in the increase of enzyme and decrease of activity. In comparison to other elastase inhibitors AFP is less inhibitory than α-1-antitrypsin (Ki=3.3×10⁻⁵ nM) but is more inhibitory than the PPE inhibitor elastatinal (Ki=5×10⁴ nM) and the previously isolated 57 kD EDP (Ki=1400 nM) (Bonnaure-Mallet et al., 1995).

The data provided in this example thus demonstrate the isolation of small EDPs that exhibit significant elastase inhibitory capacity.

In view of the above, it will be seen that the several advantages of the invention are achieved and other advantages attained.

As various changes could be made in the above methods and compositions without departing from the scope of the invention, it is intended that all matter contained in the above description and shown in the accompanying drawings shall be interpreted as illustrative and not in a limiting sense.

All references cited in this specification are hereby incorporated by reference. The discussion of the references herein is intended merely to summarize the assertions made by the authors and no admission is made that any reference constitutes prior art. Applicants reserve the right to challenge the accuracy and pertinence of the cited references.

What is claimed is:

1. An isolated and purified oligopeptide, consisting of 8 or less amino acids or amino acid mimetics and comprising the amino acid or mimetic sequence AFP.

2. The isolated and purified oligopeptide of claim 1, wherein the oligopeptide consists of 5 or less amino acids or amino acid mimetics.

3. The isolated and purified oligopeptide of claim 1, wherein the oligopeptide consists of the amino acid or mimetic sequence AFP.

4. An isolated and purified oligopeptide, consisting of the amino acid or mimetic sequence VPG.

5. An isolated and purified oligopeptide, consisting of about 20 or less amino acids or amino acid mimetics and comprising the amino acid or mimetic sequence AGIP.

6. The isolated and purified oligopeptide of claim 5, wherein the oligopeptide consists of 10 or less amino acids or amino acid mimetics.

7. The isolated and purified oligopeptide of claim 5, wherein the oligopeptide consists of the amino acid or mimetic sequence AGIP.

8. A method of inhibiting an elastolytic serine protease comprising contacting the elastolytic serine protease with an oligopeptide, wherein the oligopeptide consists of about 50 or fewer amino acids or amino acid mimetics, and wherein the oligopeptide comprises an amino acid or mimetic sequence selected from the group consisting of AFP, VPG and AGIP.

9. The method of claim 8, wherein the oligopeptide consists of 25 or fewer amino acids or amino acid mimetics.

10. The method of claim 8, wherein the oligopeptide consists of 10 or fewer amino acids or amino acid mimetics.

11. The method of claim 8, wherein the oligopeptide consists of 5 or fewer amino acids or amino acid mimetics.

12. The method of claim 8, wherein the oligopeptide comprises the amino acid or mimetic sequence AFP.

13. The method of claim 8, wherein the oligopeptide comprises the amino acid or mimetic sequence VPG.

14. The method of claim 8, wherein the oligopeptide comprises the amino acid or mimetic sequence AGIP.

15. The method of claim 8, wherein the elastolytic serine protease is a neutrophil elastase.

16. The method of claim 15, wherein the neutrophil elastase is a human neutrophil elastase.

17. The method of claim 8, wherein the neutrophil elastase is in a living mammal.

18. The method of claim 8, wherein the elastolytic serine protease is in a human.

19. The method of claim 18, wherein the mammal is suffering from a disease characterized by elevated activity of an elastolytic serine protease.

20. The method of claim 19, wherein the elastolytic serine protease is a neutrophil elastase.

21. The method of claim 20, wherein the human is suffering from, or at risk for, a condition selected from the group consisting of abdominal aortic aneurysm, adult respiratory distress syndrome, septic shock, chronic obstructive pulmonary disease, pulmonary emphysema, multiple organ failure and pulmonary hypertension.

22. The method of claim 21, wherein the human is suffering from, or at risk for, abdominal aortic aneurysm.

23. A method of detecting a disease in a mammal, wherein the disease is characterized by elevated activity of an elastolytic serine protease, the method comprising determining whether the mammal has a peptide selected from the group consisting of AFP, VPG and AGIP.

24. The method of claim 23, wherein the peptide is AFP.

25. The method of claim 23, wherein the peptide is VPG.

26. The method of claim 23, wherein the peptide is AGIP.

27. The method of claim 23, wherein the mammal is a human.

28. The method of claim 23, wherein the elastolytic serine protease is a neutrophil elastase.

29. The method of claim 28, wherein the disease is selected from the group consisting of abdominal aortic aneurysm, adult respiratory distress syndrome, septic shock, chronic obstructive pulmonary disease, multiple organ failure, pulmonary emphysema, and pulmonary hypertension.

30. The method of claim 29, wherein the disease is abdominal aortic aneurysm.

31. The method of claim 23, wherein the determination is made by testing a bodily fluid of the mammal for the peptide.

32. The method of claim 31, wherein the bodily fluid is sputum, urine or plasma.

33. A method for determining the severity of a disease in a mammal, wherein the disease is characterized by elevated activity of an elastolytic serine protease, the method comprising determining the concentration of a peptide selected from the group consisting of AFP, VPG and AGIP,
wherein a concentration of the peptide below the concentration found in a second mammal with a mild case of the disease indicates that the mammal has a severe case of the disease.

34. The method of claim 33, wherein the peptide is AFP.

35. The method of claim 33, wherein the peptide is VPG.

36. The method of claim 33, wherein the peptide is AGIP.

37. The method of claim 33, wherein the mammal is a human.

38. The method of claim 33, wherein the elastolytic serine protease is a neutrophil elastase.

39. The method of claim 33, wherein the disease is selected from the group consisting of abdominal aortic aneurysm, adult respiratory distress syndrome, septic shock, chronic obstructive pulmonary disease, pulmonary emphysema, multiple organ failure and pulmonary hypertension.

40. The method of claim 39, wherein the disease is abdominal aortic aneurysm.

41. The method of claim 33, wherein the determination is made by measuring the concentration of the peptide in a bodily fluid of the mammal.

42. The method of claim 41, wherein the bodily fluid is sputum, urine or plasma.
